(19) United States
(12) Patent Application Publication
(10) Pub. No.: US 2013/0338641 A1
(43) Pub. Date: Dec. 19, 2013

(54) Method and Apparatus for a Right-Sided Short Sheath

(71) Applicants: Seth Worley, Lancaster, PA (US); Paul Kurth, Teton Village, WY (US); Andrew Armour, Swarthmore, PA (US)

(72) Inventors: Seth Worley, Lancaster, PA (US); Paul Kurth, Teton Village, WY (US); Andrew Armour, Swarthmore, PA (US)

(73) Assignee: PRESSURE PRODUCTS MEDICAL SUPPLIES INC., Santa Barbara, CA (US)

(21) Appl. No.: 13/984,013

(22) PCT Filed: Feb. 6, 2012
(86) PCT No.: PCT/US12/23984

§ 371 Date: Sep. 5, 2013

Related U.S. Application Data
(60) Provisional application No. 61/440,250, filed on Feb. 7, 2011.

(51) Int. Cl. A61B 17/34; A61M 25/00

(52) U.S. Cl. CPC A61B 17/3421; A61M 25/0041
USPC 604/528

(57) ABSTRACT

An introducer sheath comprising a curved shape that follows the right sided vasculature when introduced through the right side subclavian vein and which stops far short of the coronary ostium in the right atrium. No attempt is made to access the coronary sinus through the sheath. The sheath establishes a fulcrum point on the upper lateral wall of the superior vena cava and comprises the ability to be removed from the implanted pacemaker lead without flipping the distal end of the sheath to cause a pull back of the pacemaker lead. The more proximal portions of the sheath extend through the superior vena cava and provide a force which biases the sheath against the lateral wall of the lower portion of the superior vena cava to establish a fulcrum or pivot point which positions the distal end of sheath at the desired location.

### RELATED APPLICATIONS

The present application is related to U.S. Provisional Patent Application Ser. No. 61/440,250, filed on Feb. 7, 2011, which is incorporated herein by reference and to which priority is claimed pursuant to 35 USC 120.

### BACKGROUND

1. Field of the Technology

The disclosure relates to the field of introducers for delivering pacing leads and catheters into the heart, and more particularly into the coronary sinus of the heart with the access point being from the right subclavian vein (alternatively the right axillary or right cephalic vein), and subsequently being removed from the vein without dislodging the lead(s) or catheter(s) delivered through the introducer.

2. Description of the Prior Art

It is a routine procedure to provide access and delivery of pacing leads and catheters into the heart from the left subclavian vein, left axillary vein, or left cephalic vein for placement of devices into the right atrium, right ventricle or coronary sinus vein. It is also common to access the heart from the right subclavian vein when left sided access is not practical.

The problem of the prior art is to provide a cardiac introducer which can reliably and easily deliver pacing leads and catheters into the coronary sinus of the heart, from the right side (preferably from the right subclavian vein, or alternatively the right axillary or right cephalic vein), and subsequently removed without dislodging a permanent pacing lead delivered through the introducer. The distal end of a conventional left-side introducer 18 as shown in FIG. 2 can be successfully used for lead implantation through the right side subclavian vein into the coronary sinus 20 only with great difficulty and not with good repeatability or ease. The success of such an implantation depends critically on the skill of the cardiologist, the particular cardiac anatomy of the patient and luck. Subsequent removal of the conventional left side introducer 18 frequently dislodges the pacing lead as it is being removed from the right subclavian vein.

An additional problem is that the junction in many, if not most, patients between the right subclavian vein and the superior vena cava can approach that of a right angle. A straight introducer or catheter being inserted through this vascular junction can then easily be kinked, which renders it subsequent use as a telescopic introducer impractical or impossible.

Further, introducers which are employed for inserting a catheter or inner introducer into the right atrium through the right subclavian vein should have a J shaped distal portion with sufficient curvature to direct the catheter or inner introducer toward the coronary ostium in order to facilitate implantation of a cardiac catheter into the coronary sinus. As the conventional introducer is removed from the pacemaker lead the bias built into the curvature of the steerable introducer in combination with the typical curvatures encountered in the human right subclavian-to-superior-vena-cava path are such that the introducer is unavoidably torqued or rotates. The rotation is actually impulsive and the distal end of the conventional introducer flips in a sudden unavoidable rotation as the curved introducer is pulled through the right subclavian-to-superior-vena-cava junction. This in turn causes the pacemaker lead, which has been implanted into the coronary ostium or coronary sinus, to pull out at least to some degree so that the intended implanted position is lost.

A right sided introducer to the coronary sinus was previously developed by one of the inventors in the present application and is set forth within U.S. application publication 2006/0161177. This prior art introducer had as its object, whether used alone or with a dilator or proximal outer introducer, the direct access to the coronary sinus of the heart. The prior art introducer as shown in FIG. 2a included a first proximal curve 22 approximating a right angle or with a few tens of degrees of a right angle and ending ultimately in a distal reverse curve 30. Again it must be emphasized, as stated in the abstract and as generally taught throughout the publication “[t]he straight and curved sections collectively have a length and shape to dispose the distal tip at or near the coronary sinus when accessed from the right subclavian vein.” The defining design goal of this introducer was that it successfully cannulate the coronary sinus of the heart from a right side access. Significantly, this introducer did not establish a fulcrum or purchase point on the lateral wall by which an inner telescopic introducer could then be steered into the coronary sinus. Secondly, this prior art introducer also did not avoid, when removed, applying a twisting torque applied to a pacemaker lead disposed through it. Like other prior art introducers, it flipped the distal reverse curve 30 of the introducer as it was pulled out of the superior vena cava and as a result displaced the implanted position of the pacemaker lead disposed through it.

The prior art introducer was found to be unacceptably difficult to steer and manipulate to achieve access from the right side subclavian vein into the coronary sinus and thus was regarded as a difficult-to-use device. There was nothing in the teachings relating to the prior art introducer which lead to or gave any reason how to alter its design to gain an acceptable ability to steer and manipulate a coronary catheter to access the coronary sinus from the right side subclavian vein. In fact, it was surprising to find that elements of an unsuccessful design to provide reliable cannulation to the coronary sinus could be altered in some way to achieve successful cannulation to the coronary sinus in the manner disclosed below.

### BRIEF SUMMARY

The present invention overcomes the shortcomings of the prior art with an introducer sheath curve shape that follows the right sided vasculature when introduced through the right side subclavian vein and which stops far short of the coronary ostium in the right atrium and which establishes a fulcrum point against the lateral wall of the right atrium. No attempt is made to access the coronary sinus through introducer 11. Instead a second introducer (not shown) is telescopically disposed through introducer 11 to make the final access as described below.

The illustrated embodiments of the invention also include an introducer for right side access which have a first proximal curve approximating a right angle or with a few tens of degrees of a right angle and ending ultimately in a distal reverse curve. However, the embodiments of the introducer disclosed below have a predetermined length and are shaped to have one or both of the characteristics described above, namely establishment of a fulcrum point on the lateral wall of the right atrium and/or the ability to be removed from the implanted pacemaker lead without flipping the distal end of the introducer to cause a pull back of the pacemaker lead. None of the embodiments of the introducer of the invention will by itself or with the use of a dilator or an outer proximal introducer directly cannulate the coronary sinus in any remote sense as was the object and result in the case of the prior art introducer.

The invention includes a cardiac outer introducer with a proximal and distal end. The introducer is for the insertion of a catheter through a right subclavian vein, superior vena cava and right atrium into a heart having a coronary sinus ostium, leading to a coronary sinus and having a right ventricle. The introducer includes a proximal hub or valve coupled to its proximal end with a sheath coupled to it, the introducer having a length as determined by a size of the heart to sufficiently access the superior vena cava or right atrium from the right side subclavian vein. The sheath has a first portion with a first radius of curvature extending from the proximal end of the sheath to a transition point on the sheath, and a second portion with a second radius of curvature opposite to the first radius of curvature extending from the transition point to the distal end or a straight portion extending from the transition point to the distal end. The distal end of the sheath is oriented toward the tricuspid valve or coronary sinus ostium in order to provide a launch point when inserted into the heart for the introduction of a lead or catheter into the coronary sinus through the coronary sinus ostium or into the right ventricle.

In one embodiment, the first portion of the introducer comprises two curved subportions, each with a corresponding radius of curvature of the same sense and a straight portion between the two curved subportions.

In another embodiment, the second radius of curvature of the second portion of the introducer is 0.5 to 1.5 inch (1.27-3.81 cm).

The introducer further includes a straight proximal portion that is disposed between the proximal hub and the first portion, the first portion being followed by an adjacent distal straight portion inclined relative to the straight proximal portion at an angle of 30-180°.

In another embodiment, the second portion of the introducer has a projection at its distal end inclined relative to the proximal straight portion at an angle of 0-180°.

In one specific embodiment, the introducer has a total length of 18-35 centimeters.

In yet another embodiment, the introducer has a straight portion extending from the first portion to the distal end of the sheath.

The sheath of the introducer also includes means for bending a conventionally shaped left-sided CS access catheter through the right subclavian vein, superior vena cava, and right atrium to make the left sided CS catheter capable of being manipulated as though it were placed from the left subclavian vein.

In one embodiment, the distal end of the sheath of the introducer does not cannulate or extend to the coronary ostium.

In yet another embodiment, the sheath of the introducer includes specific means for not applying a torque to a pacemaker lead or other catheter disposed through it such that, when the sheath is removed from the pacemaker lead or other catheter, the distal end of the sheath does not turn or flip causing an implanted pacemaker lead or other catheter to be pulled out of the coronary sinus or displaced from its implanted position.

The invention also provides for a cardiac outer introducer for the insertion of a catheter through a right side subclavian vein, superior vena cava and right atrium into a heart having a coronary sinus ostium leading to a coronary sinus. The introducer includes a sheath with a proximal hub or valve coupled to its proximal end, the sheath having a length extending to a distal end. The length of the sheath is determined by the size of the heart in which it is to be inserted and such that it is sufficient to access the superior vena cava or right atrium of the heart from the right side subclavian vein. The sheath itself includes at least a first curve with at least a first radius of curvature extending from the proximal end on the sheath and a second curve with a second radius of curvature opposite to the first radius of curvature extending from the first curve to a distal end of the sheath. The first curve of the sheath is shaped to follow the contour of the subclavian vein and superior vena cava when implanted into the heart. The second curve of the sheath is shaped to contact the lateral wall of the superior vena cava or right atrium when implanted into the heart so that the distal end of the sheath is oriented toward the tricuspid valve or coronary sinus ostium to provide a launch point for introduction of a catheter into the coronary sinus through the coronary sinus ostium or into a right ventricle of the heart.

In one particular embodiment, the sheath of the introducer has a length and a second curve which are arranged and configured to assist a steerable distally curved inner introducer to access the coronary ostium, through which inner introducer a pacemaker lead or catheter is telescopically disposable into the coronary sinus. The second curve of the sheath and curvature of the steerable distally curved inner introducer are such that sequential removal of neither the inner introducer nor the sheath from the pacemaker lead or catheter causes a substantial displacement of the pacemaker lead or catheter from its intended implanted position within the coronary sinus.

The invention further provides a method of using an outer sheath for right sided access to the heart of a patient, the method including disposing the sheath through the right subclavian vein, superior vena cava, and right atrium of the heart and establishing at least one fulcrum point with the sheath against a lateral surface within the superior vena cava or right atrium. The sheath is prebiased with a first radius of curvature which is positioned within the superior vena cava and prebiased with a second radius of curvature opposite to the first radius of curvature distally from the first radius of curvature. The distal end of the sheath is orientated toward the tricuspid valve or coronary sinus ostium to provide a launch point for introduction of a lead or catheter into the coronary sinus through the coronary sinus ostium or into a right ventricle.

In one embodiment, the method further includes biasing the sheath with the first and second radii of curvature prior to disposing the sheath and then inclining the first curvature of the sheath 30-180° relative to a proximal straight portion of the sheath.

In another embodiment, the method further includes biasing the sheath with the second radius of curvature opposite to the first radius of curvature and inclining the sheath at the second radius of curvature 0-180° relative to a proximal straight portion of the sheath.

In yet another embodiment, the method further includes establishing a second fulcrum point with the sheath against a lateral surface within the superior vena cava or right atrium.

In yet another embodiment, the method further includes inserting a conventionally shaped left sided CS access catheter through the sheath and manipulating the left sided CS access catheter through the right subclavian vein, superior vena cava, and right atrium as though it were placed from the left subclavian vein.

The method in another embodiment further comprises preventing the sheath from cannulating or extending to or near the coronary ostium and disposing a pacemaker lead or catheter through the sheath to access the coronary sinus through the coronary sinus ostium or to access a right ventricle. The sheath may be removed from the pacemaker lead or catheter without displacing the pacemaker lead or catheter from its intended implanted position within the coronary sinus. Furthermore, the method further includes removing the sheath from the pacemaker lead or catheter without applying a torque to the pacemaker lead or catheter or turning or flipping the distal end of the sheath.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

The specification contains at least one drawing executed in color. Copies of this patent or patent application publication with color drawing(s) will be provided by the Office upon request and payment of the necessary fee.

FIG. 1 is a side plan view of a right sided short sheath or cardiac sheath, which includes a separable valve or hub and a curved introducer used to access the right atrium of a heart from the right subclavian vein.

FIG. 2 is a cross sectional diagram of the heart depicting both a left sided introducer of the prior art and the ride sided introducer sheath of the current invention accessing the right atrium.

FIG. 3 is a plan view illustration of a plurality of different embodiments where a pre-biased shape of the sheath is characterized by a distal portion with reverse curvature such as also illustrated in FIG. 1.

FIG. 4 is a parameterized diagram of the shape of an embodiment of the sheath, enumerated by points 101-127.

FIG. 5 is a plan view illustration of an alternative embodiment of the pre-biased sheath seen in FIG. 1 and depicting the specific angles of curvature for each portion of the sheath.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

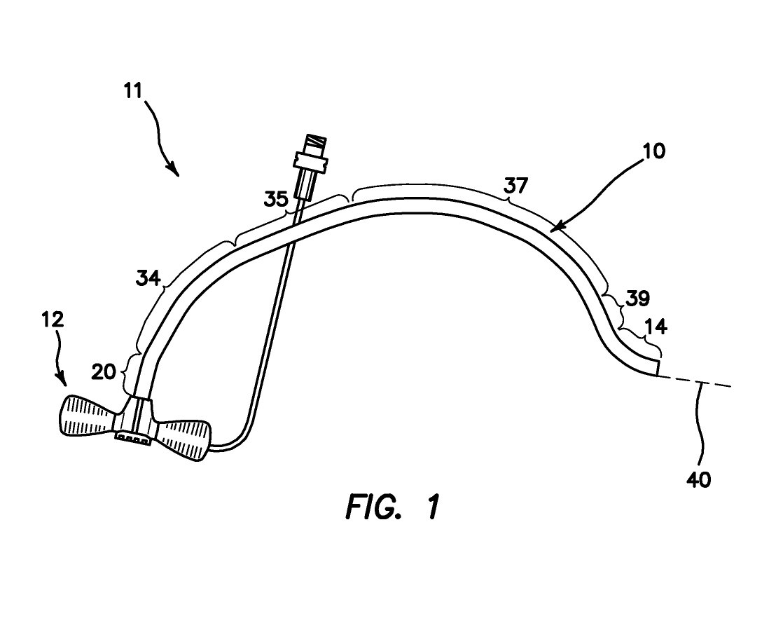

FIG. 1 is a side plan view of a prebiased, right sided short sheath or cardiac introducer 11, which includes a separable valve or hub 12 and a curved sheath 10 used to access the right atrium from the right subclavian vein. Sheath 10 is a separable introducer 11 as is well known to the art, i.e. it may be longitudinal separated by splitting, tearing, cutting or any other means of separation so that it may be removed over the hub or other obstruction of a pacemaker lead or other elongate tool that may be telescopically disposed through it. Sheath 10 may be longitudinally separated by use of molecular lines of weakening in the walls of the sheath 10, grooves or indentations defined into sheath 10 or any other means now known or later devised for allowing longitudinal separation. However, the scope of the invention also includes the possibility that sheath 10 may be torqueable by being braided or otherwise reinforced so that the use of a cutting tool may then be required into order to longitudinally separate it. However, it is to be understood that sheath 10 and valve 12 may not be separable, e.g. in the case where the pacing lead or instrument disposed through introducer 11 is isodiametric allowing for removal of introducer 11 over the proximal end of the instrument or pacing lead.

In the illustrated embodiment of FIG. 1 the throw or horizontal distance from portion 20 to distal end portion 14 as seen in the plane of the drawing is approximately 6.5 inches (16.5 centimeters). The horizontal distance in FIG. 1 from portion 20 to the beginning of the distal curve 14 is approximately 5.6 inches (14.2 centimeters). The height in FIG. 1 of the cumulative curve 34, 35, 37 is approximately 2.1 inches (5.3 centimeters) as measured from the point (peak) furthest away from a horizontal projection line from the distal end of sheath 10. The vertical distance in FIG. 1 from the peak to the beginning of distal curve 14 is approximately 1.6 inches (4 centimeters). In other embodiments the sizes and shapes may vary according the teachings of the invention.

Access through the right subclavian vein is shorter though less direct than from the left side subclavian vein, with a greater curve in the right innominate vein. A conventional distal hook (90 degree curve) catheter, not shown, is then introduced through sheath 10 into the coronary sinus 6 of the heart opening into the right atrium 16 for pacemaker lead implant into the sinus system of the heart or other cardiac sinus endovascular intervention. The two stage introduction of the hook catheter from a lateral platform or launch site at the distal end of sheath 10 allows for better control of and success in implantation of the hook catheter into the coronary sinus. The directional positioning of sheath 10 in combination with the torqueable nature of the hook catheter provides the necessary combination of positioning, control and torque in the right atrium 16 to mimic the well known technique used to access the coronary sinus 6 from the left side.

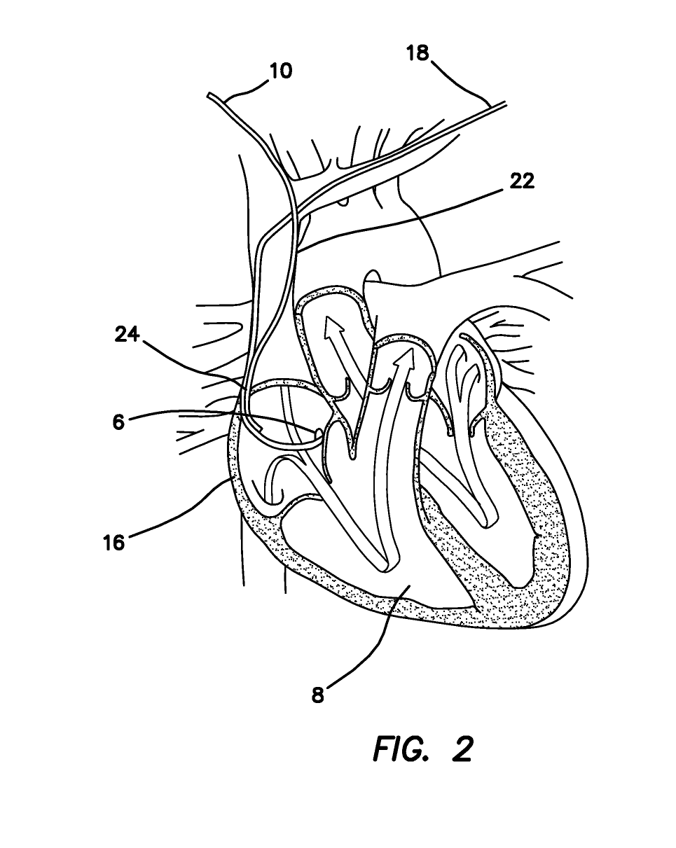

Sheath 10 is curved or manufactured with a pre-bias according to the dimensions as shown in FIG. 1 to appropriately fit or contact the average anatomical shape of the superior vena cava and upper lateral wall shape of the right atrium 16 to provide for two purchase points 22 and 24 as shown in FIG. 2 to insure appropriate positioning of the distal end of sheath 10. Sheath 10 normally lies in a plane when exterior to the heart. It is to be understood that the sizes and angles of inclinations may be varied according to the teachings of the invention to accommodate different classes of patients without departing from the spirit and scope of the invention. For example, a smaller version of introducer 11 sized for adults would be provided prebiased according to the teachings of the invention to generally accommodate the anatomy of infants or children. Still further, a larger version of introducer 11 sized for adults would be provided prebiased according to the teachings of the invention to generally accommodate the anatomy of patients having enlarged hearts.

It is also to be noted in FIG. 1 that there is a first proximal curved portion 34 in sheath 10 which is at a location in sheath 10 so that it is positioned at the junction of the right subclavian vein and superior vena cava when the sheath 10 is fully inserted through the right subclavian vein into the right atrium 16. Bend or curve 34 is illustrated in FIG. 1 begins with a first approximately straight run 20 of the sheath distally from the distal end of the valve or hub 12 and then curving clockwise to lead to a second approximately straight portion 35 which is included relative to portion 20 by approximately 120°, but it is entirely within the embodiments of the invention that curve 34 may assume any inclination within the range of 30° to 180° to traverse the junction of the right subclavian vein and superior vena cava without kinking. The angle or inclination which will be employed in curve 34 will depend on the stiffness of the material out of which sheath 10 is composed, whether or not sheath 10 is reinforced, its wall thickness as well as the specific anatomy of the patient. Depending on all relevant variables involved, the angle of curve 34 is empirically determined to that which in the specific embodiment and application is not subject to kinking when sheath 10 is disposed through the junction of the right subclavian vein and superior vena cava.

It is also to be noted in FIG. 1 that there is a second distal curved portion 14 in sheath 10 which is at a location in sheath 10 so that it is positioned within the right atrium 16 when the sheath 10 is fully inserted. A third curve 37 is prebiased or defined in sheath 10 distally from straight portion 35 and terminates with a distal straight portion 39 before transitioning to distal curve 14. Bend or curve 14 is illustrated in FIG. 1 as having a projection 40 at its distal end inclined relative to straight portion 39 by approximately 130° with a radius of 1″, but it is entirely within the embodiments of the invention that curve 14 may assume any angle between projection 40 at its distal end relative to straight portion 39 within the range of 0° to 180° and a radius of 0.5″ to 1.5″ to sufficiently direct the inner catheter towards the coronary sinus ostium 6 yet without inducing a flipping torque on the distal end of the sheath 10 when it is removed from the pacemaker lead disposed through it. The angle or inclination which will be employed in curve 14 will depend on the stiffness of the material out of which sheath 10 is composed, whether or not sheath 10 is reinforced, its wall thickness as well as the specific anatomy of the patient. Depending on all relevant variables involved, the angle and radius of curve 14 is empirically determined to that which in the specific embodiment and application is not subject to kinking or flipping when introducer 11 is removed through the junction of the right subclavian vein and superior vena cava.

It is to be understood that the term “first curve” in this specification and claims is used to include in the illustrated embodiment at least curve 34, but also can be understood to include straight portion 35, curved portion 37 and straight portion 39 or other curves in other embodiments as a compound or complex curve with not one but with corresponding multiple radii of curvature. A straight portion can be thought of as having an infinite radius of curvature. Thus, wherever the term, “first curve” is used it may include a single simple curve or a multiple number of curved and straight curves in any combination with different or the same radii of curvature. However, the “first curve” has a radius or radii of curvature which are all in the same sense, i.e. all in the same direction as contrasted with the “second curve” 14 which as a radius of curvature in a sense or direction opposite to that of the “first curve”.

It is to be understood that the prebiased ranges of shape are those profiles which introducer 11 would tend to assume if laid flatly on a planar surface without being subject to outside forces. When implanted into a heart, introducer 11 is flexible and pliable so its prebiased form will readily conform to the forces and shapes of the vascular system and heart. However, whenever the anatomy of the vascular system and heart allows introducer 11 to assume its prebiased configuration, it will tend to do so.

Sheath 10 is led into the right atrium 16 as shown in FIG. 2 to serve as a platform for entry or introduction means for the hook introducer. Also shown for purposes of comparison only is the simultaneous introduction of a conventional left side introducer 18. FIG. 2 shows that the distal end of sheath 10 is the same or similar to the path of introduction of conventional left side introducer 18, which is shown as leading up to and/or into the coronary sinus 6.

The right sided introducer 11 or sheath 10 curves a standard shaped left sided CS access catheter in such a way that makes the CS catheter bend and be able to be manipulated as though it was being placed from the left side.

In one embodiment a second inner catheter or introducer (not shown) is telescopically disposed into sheath 10 and extends from its distal end and is directed by sheath 10 into the coronary ostium 6 of the heart. This inner introducer typically is torqueable so that it may be rotated out of plane with respect to the plane generally defined by sheath 10 to provide a means for steering and a means to accommodate varying scales of anatomies found in patients.

In general, sheath 10 as shown in FIG. 2 contacts the lateral wall of the lower portion of the superior vena cava at point 22 or possibly the upper portion of the right atrium to gain a fulcrum or pivot point by which sheath 10 is positioned so that its distal end is generally supported in a position to direct the inner catheter toward the coronary ostium 6. The more proximal portions of sheath 10 extending through the superior vena cava provide a force which biases sheath 10 against the lateral wall of the lower portion of the superior vena cava at point 24 or possibly the upper portion of the right atrium to establish this fulcrum or pivot point and hence the purchase which positions the distal end of sheath 10 at the desired location. However, it must be kept in mind that the scope of the invention allows for the embodiment that does not require contact of the sheath 10 against the lateral wall of the lower portion of the superior vena cava at point 24 or possibly the upper portion of the right atrium, but allows sheath 10 to float freely in this position. The bias and shape of sheath 10, and particularly its distal portion, can be sufficient in this embodiment to provide sufficient support and direction to the inner catheter to direct it toward the coronary ostium 6.

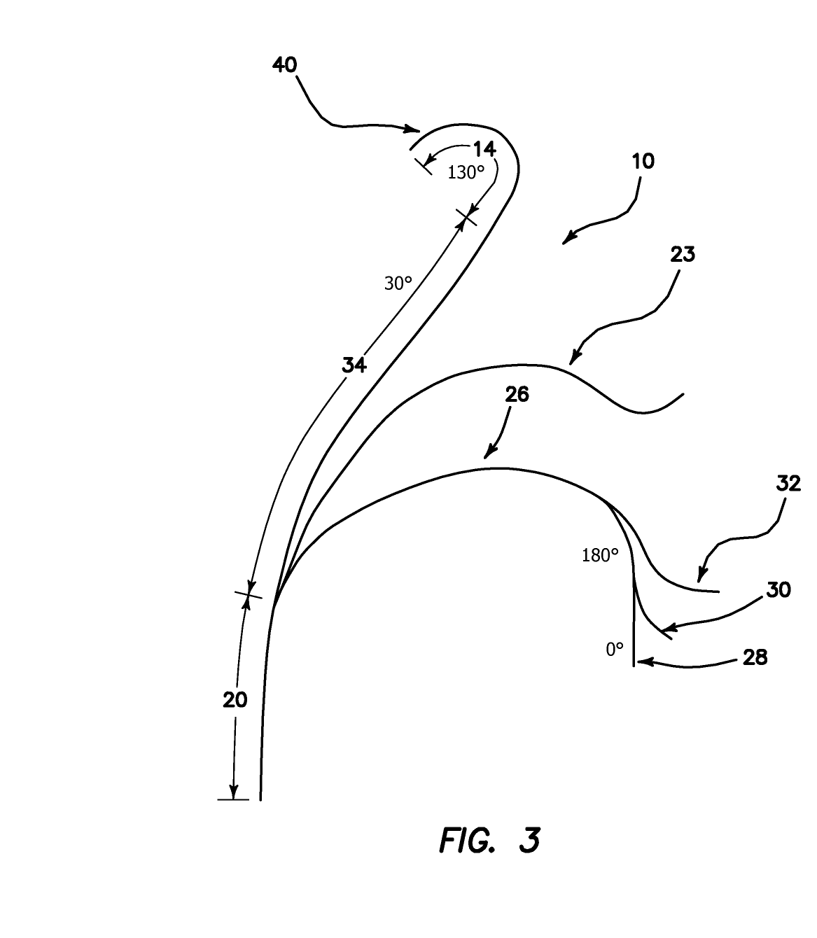

FIG. 3 is a plan view illustration of a plurality of different embodiments where a pre-biased shape of the sheath 10 is characterized by a distal portion with reverse curvature such as also illustrated in FIG. 1. For example, in the intermediate or first proximal curved portion 34 between a proximal straight portion 20 and the second distal curved portion 14 of sheath 10, the embodiments begin with a smooth transition of curved portion 34 to an inclination of approximately 30° from the line of proximal straight portion 20, i.e. straight portion 35 is inclined to straight portion 20 by approximately 60°, but this inclination may be anywhere in the range of 30-180°. The second distal curved portion 14 then shows a reverse curvature with brings the distal tip 40 of sheath 10 to an inclination where it points in a direction approximately 130 degrees away from the lead in direction from the first proximal curved portion 34. However, the second distal portion 14 may have a relative inclination with respect to the proximal straight portion 20 at any angle in the range of 0-180°.

The first proximal curved portion 34 may assume other inclinations from proximal portion 20 as shown in FIG. 3 by the position denoted by sheath 10 assumed by configuration 23 or 26. In the embodiment of configuration 26, the distal end of the first proximal curved portion 34 assumes a direction approximately 180 degrees from that assumed by the lead-in direction of proximal straight portion 20. However, even in configuration 26 the distal curved portion 14 may assume a variety of configurations, such as that shown by configuration 28 where it is straight or nearly straight, or where it assumes a reverse curvature of varying degrees as shown by configurations 30 or 32. It is to be expressly understood that the first proximal curved portion 34 may assume other positions relative to the straight proximal portion 20 other than what is explicitly shown in FIG. 3 without departing from the original spirit and scope of the invention. Furthermore, it is also well within the scope of the current invention to have the second distal curved portion 14 assume a variety of positions relative to the first proximal curved portion 34 other than what is shown and described above. The embodiments shown in FIG. 3 are meant to be for illustrative purposes only and should constrain or limit the number of configurations capable by the current invention.

One of the attributes of the illustrated embodiments of the invention as shown in FIG. 3 is that the bias manufactured into sheath 10 is that when the second or inner catheter is telescopically disposed through sheath 10, the torquing or flipping of the inner catheter does not occur with the consequent loss of implanted position of the pacemaker lead as was the case with the prior art. To facilitate this performance, the inner catheter is flexible or unbiased except for its distal portion which extends from sheath 10. Only the J shaped tip of the inner catheter has a predetermined bias fabricated into it as must be the case in order to be able to steer it into the coronary ostium 6. However, flexibility of the pacemaker lead is not sufficient to prevent the conventional right sided introducer from flipping its distal end when it is withdrawn from the pacemaker lead, thereby causing the pacemaker lead to be pulled out from position. In the illustrated embodiment the length of the reverse curve on the distal portion of sheath 10 is short enough such that when sheath 10 is pulled out through the right subclavian-to-superior-vena-cava junction, the reversed curved distal end of sheath 10 does not flip or torque the pacemaker lead so much that it is pulled out of position. In the illustrated embodiment the range of lengths of the second distal curve 14 on the distal portion of sheath 10 is preferably 2 to 6 cm. Similarly, when the pacemaker lead is disposed through the inner catheter and implanted through the coronary ostium 6 into the coronary sinus and then the inner catheter pulled back or removed, there is no or very minor torque or twisting applied to the pacemaker lead by the biased end of the inner catheter to cause the pacemaker lead to be pulled out of position. Again, when the sheath 10 is then removed from the flexible pacemaker lead after removal of the inner catheter, any unavoidable rotation of sheath 10 is also not transmitted to the distal implanted end of the pacemaker lead. The reverse curvature needed to reliably guide the pacemaker lead to the coronary ostium 6 is thus effectively divided between the inner catheter and the sheath 10 so that torquing or flipping on removal through the right angled curve of the right subclavian-to-superior-vena-cava junction does not occur.

It must be noted again that the illustrated embodiments of the invention comprise a sheath 10 which is not itself intended to provide direct access to the coronary sinus through the coronary ostium 6. As stated above, a second introducer deployed through sheath 10 is employed to cannulate the coronary sinus 6. This difference distinguishes sheath 10 from the embodiments of conventional introducers disclosed in the cited art, each of which is capable of accessing the coronary sinus. While the position of the second distal curve 14 for any one of the illustrated embodiments will necessarily vary in the right atrium 16 according to the anatomy of the patient in which it is deployed, its intended disposition is that shown in FIG. 2. For example, if an adult-sized sheath 10 where disposed in a child, it might well turn out in some cases that the distal curve would approach the coronary ostium 6 simply due to the fact that the child's heart is smaller and the adult-sized sheath 10 is oversized for that patient. Nevertheless, as disclosed above the radius of curvature of the second distal curve 14 is in the range of 0.5 to 1.5 inch (1.27-3.81 cm), which is outside of the disclosed ranges for the reverse distal curve used in the family of introducers disclosed in the prior art such as U.S. application publication 2006/0161177. For example, in FIG. 7 of the cited art, the radius of curvature of the distal reverse curve is disclosed as 2.75 inches (6.985 cm), which is necessary in order to achieve direct cannulation. The distinction is that with the size of the radii of the distal curves of conventional introducers withdrawal of the introducer is sure to apply a torque to the implanted pacemaker lead and likely flip the distal end of the introducer and displace the pacemaker lead. The smaller radius of curvature employed in the right-side sheaths 10 of the illustrated embodiments are not prone to flip the distal end and displace the lead, and particularly so when the sheath 10 is properly sized for the patient in which it is used.

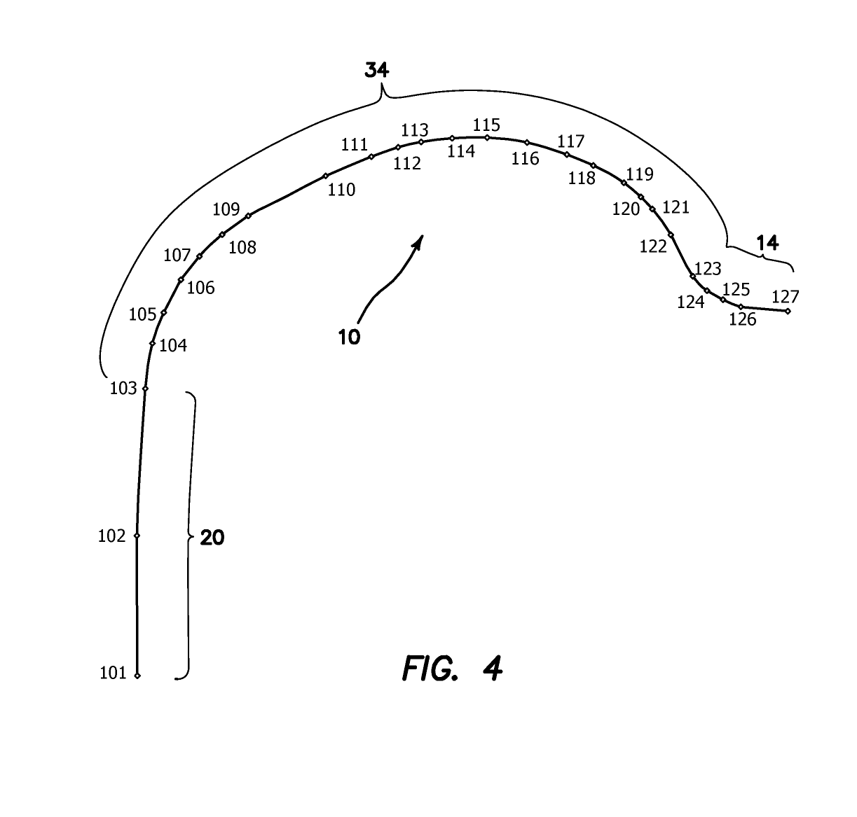

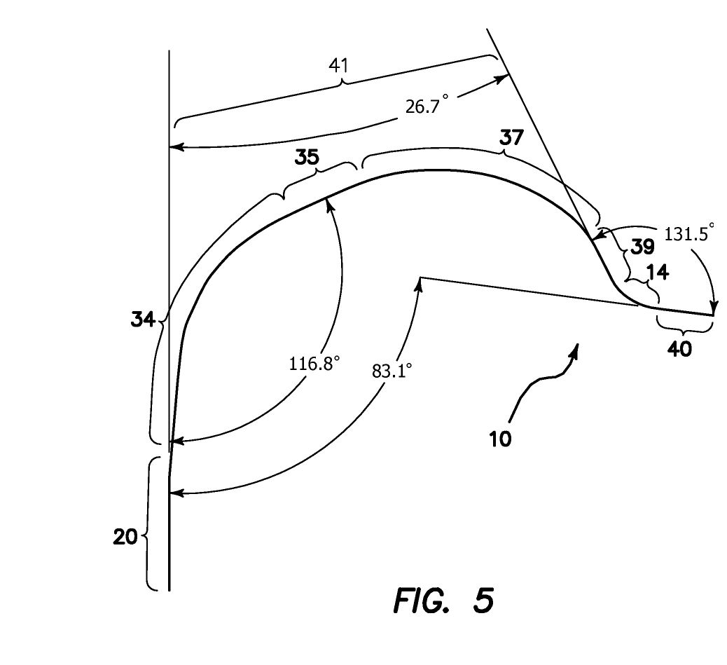

FIG. 4 is a parameterized diagram of the shape of an embodiment of the sheath 10, enumerated by points 101-127. Sheath 10 in the illustrated embodiment has a planar form, so that it can be conveniently specified by the x and y coordinates of the parameterized points. This parameterization is shown below in Table 1. FIG. 4 visually demonstrates along with the values of points 123-127 in Table 1 that the radius of curvature of the second distal curve 14 is substantially reduced from that shown in the introducers of the prior art. Thus even if the second distal curve 14 were somehow positioned near or in the coronary OS, the direction of a pacemaker lead disposed through it would not likely be correctly oriented for entry into the coronary sinus 6. The shape of sheath 10 of FIG. 4 is further illustrated by the angulations of sheath 10 as shown in FIG. 5. It can be graphically seen that the cumulative effect of the first proximal curve 34 and the second distal curve 14 is such that the angle between a tangent or projection 40 of the distal end at point 127 with respect to the straight proximal portion 20 is approximately 83°. The cumulative effect of curves 34 and 37 are such that the inclination between straight portions 20 and 39 is approximately 26.7°. The effect of curve 34 is such that the inclination between straight portions 20 and 35 is approximately 116.8°. The effect of curve 14 is such that the inclination between straight portion 39 and projection 40 is approximately 131.5°. Straight proximal portion 20 is disposed in and hence approximately aligned with the direction of the right subclavian vein with the result that the bias of the various bends and curves of sheath 10, as might be further altered by the anatomy, will direct the distal end of the sheath 10 83° with respect to the line of the right subclavian vein or generally in a right angled direction thereto.

TABLE 1
Right Sided Curve Data Points (in)

| Point | x | y |
|---|---|---|
| 101 | 0 | 0 |
| 102 | 0 | 1.408 |
| 103 | 0.094 | 2.922 |
| 104 | 0.169 | 3.378 |
| 105 | 0.287 | 3.683 |
| 106 | 0.462 | 4.023 |
| 107 | 0.646 | 4.262 |
| 108 | 0.877 | 4.484 |
| 109 | 1.142 | 4.67 |
| 110 | 1.929 | 5.068 |
| 111 | 2.391 | 5.264 |
| 112 | 2.661 | 5.352 |
| 113 | 2.897 | 5.406 |
| 114 | 3.212 | 5.444 |
| 115 | 3.568 | 5.451 |
| 116 | 3.976 | 5.395 |
| 117 | 4.382 | 5.267 |
| 118 | 4.649 | 5.163 |
| 119 | 4.961 | 4.975 |
| 120 | 5.128 | 4.84 |
| 121 | 5.247 | 4.719 |
| 122 | 5.429 | 4.456 |
| 123 | 5.65 | 4.016 |
| 124 | 5.792 | 3.87 |
| 125 | 5.955 | 3.777 |
| 126 | 6.137 | 3.707 |
| 127 | 6.608 | 3.65 |

It is to be understood that in the preferred embodiments the various curves and straight portions, which comprise the shape of sheath 10, smoothly transition into each other so that there are no kinks or sharp corners. In mathematical terms, this property would be defined by stating that the first derivative of the shape of sheath 10 is continuous at each point in space.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

For example, the scope of the invention is not limited to the embodiments illustrated in the figures, but include an application of the concept of the invention in a right sided sheath, which is adaptable to a wide range of differing scales of patient anatomy, which is highly variable and for which no closed specification could possibly include all patients. Typically, sheath 10 will have a length in the range of 18-35 cm with most embodiments in the subrange of 23-25 cm. The first proximal curved portion 34 may be inclined relative to the proximal straight portion 20 at angles in the range of 30-180 degrees. The second distal curved portion 14 may be inclined relative to the first proximal curved portion 34 at angles in the range of 0-180 degrees, with the subrange of 120-150 degrees being the most common. The angular inclination of the first proximal curved portion 34 relative to the proximal straight portion 20 is within the range of 30-180 degrees with the subrange of 150-175 degrees being most common.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A cardiac outer introducer with a proximal and distal end, the introducer for insertion of a catheter through a right subclavian vein, superior vena cava and right atrium into a heart having a coronary sinus ostium leading to a coronary sinus and having a right ventricle comprising:
a proximal hub or valve coupled to the proximal end of the introducer, a sheath coupled to the hub or valve having a length as determined by a size of the heart sufficient to access the superior vena cava or right atrium of the heart from the right side subclavian vein, the sheath having a first portion with a first radius of curvature extending from the proximal end of the sheath to a transition point on the sheath, and a second portion with a second radius of curvature opposite to the first radius of curvature extending from the transition point to the distal end or a straight portion extending from the transition point to the distal end;
wherein the distal end of the sheath is oriented toward the tricuspid valve or coronary sinus ostium to provide a launch point when inserted into the heart for introduction of the catheter into the coronary sinus through the coronary sinus ostium or into the right ventricle.

2. The introducer of claim 1 where the first portion comprises two curved subportions, each with a corresponding radius of curvature of the same sense and a straight portion between the two curved subportions.

3. The introducer of claim 1 where the second radius of curvature of the second portion is 0.5 to 1.5 inch (1.27-3.81 cm).

4. The introducer of claim 1 further comprising a straight proximal portion disposed between the proximal hub and the first portion and where the first portion is followed by an adjacent distal straight portion inclined relative to the straight portion at an angle of 30-180°.

5. The introducer of claim 4 where the second portion has a projection at its distal end inclined relative to the proximal straight portion at an angle of 0-180°.

6. The introducer of claim 1 comprising a total length of 18-35 centimeters.

7. The introducer of claim 1 further comprising a straight portion extending from the first portion to the distal end of the sheath.

8. The introducer of claim 1 where the sheath comprises means for bending a conventionally shaped left-sided CS access catheter through the right subclavian vein, superior vena cava, and right atrium to make the left sided CS catheter capable of being manipulated as though it were placed from the left subclavian vein.

9. The introducer of claim 1 where the distal end of the sheath does not cannulate or extend to or near the coronary ostium.

10. The introducer of claim 1 where the sheath comprises means for not applying a torque to a pacemaker lead or other catheter disposed through it such that, when the sheath is removed from the pacemaker lead or other catheter, the distal end of the sheath does not turn or flip causing an implanted pacemaker lead or other catheter to be pulled out of the coronary sinus or displaced from its implanted position.

11. A cardiac outer introducer for insertion of a catheter through a right side subclavian vein, superior vena cava and right atrium into a heart having a coronary sinus ostium leading to a coronary sinus comprising:
a proximal hub or valve coupled to a proximal end of the sheath; and
a sheath having a length extending to a distal end, the length as determined by a size of the heart being sufficient to access the superior vena cava or right atrium of the heart from the right side subclavian vein, the sheath comprising:
at least a first curve with at least a first radius of curvature extending from the proximal end on the sheath;
a second curve with a second radius of curvature opposite to the first radius of curvature extending from the first curve to a distal end of the sheath,
wherein the first curve is shaped to follow the contour of the subclavian vein and superior vena cava when implanted into the heart, the second curve being shaped to contact the lateral wall of the superior vena cava or right atrium when implanted into the heart so that the distal end of the sheath is oriented toward the tricuspid valve or coronary sinus ostium to provide a launch point for introduction of the catheter into the coronary sinus through the coronary sinus ostium or into a right ventricle of the heart.

12. The introducer of claim 11 where the length of the sheath and the second curve is arranged and configured to assist a steerable distally curved inner introducer to access the coronary ostium, through which inner introducer a pacemaker lead or catheter is telescopically disposable into the coronary sinus, the second curve of the sheath and curvature of the steerable distally curved inner introducer being such that sequential removal of neither the inner introducer nor the sheath from the pacemaker lead or catheter causes a substantial displacement of the pacemaker lead or catheter from its intended implanted position within the coronary sinus.

13. A method of using an outer sheath for right sided access to the heart of a patient comprising:
disposing the sheath through the right subclavian vein, superior vena cava, and right atrium of the heart;
establishing at least one fulcrum point with the sheath against a lateral surface within the superior vena cava or right atrium;
where the sheath is prebiased with a first radius of curvature which is positioned within the superior vena cava;
where the sheath is prebiased with a second radius of curvature opposite to the first radius of curvature distally from the first radius of curvature; and
orientating the distal end of the sheath toward the tricuspid valve or coronary sinus ostium to provide a launch point for introduction of a catheter into the coronary sinus through the coronary sinus ostium or into a right ventricle.

14. The method of claim 13 further comprising biasing the sheath with the first and second radii of curvature prior to disposing the sheath and inclining the first curvature of the sheath 30-180° relative to a proximal straight portion of the sheath.

15. The method of claim 13 further comprising biasing the sheath with the second radius of curvature opposite to the first radius of curvature and inclining the sheath at the second radius of curvature 0-180° relative to a proximal straight portion of the sheath.

16. The method of claim 13 further comprising establishing a second fulcrum point with the sheath against a lateral surface within the superior vena cava or right atrium.

17. The method of claim 13 further comprising inserting a conventionally shaped left sided CS access catheter through the sheath and manipulating the left sided CS access catheter through the right subclavian vein, superior vena cava, and right atrium as though it were placed from the left subclavian vein.

18. The method of claim 13 further comprising preventing the sheath from cannulating or extending to or near the coronary ostium and disposing a pacemaker lead or catheter through the sheath to access the coronary sinus through the coronary sinus ostium or to access a right ventricle.

19. The method of claim 18 further comprising removing the sheath from the pacemaker lead or catheter without displacing the pacemaker lead or catheter from its intended implanted position within the coronary sinus.

20. The method of claim 18 further comprising removing the sheath from the pacemaker lead or catheter without applying a torque to the pacemaker lead or catheter or turning or flipping the distal end of the sheath.
